(19) United States
(12) Patent Application Publication
(10) Pub. No.: US 2006/0166215 A1
(43) Pub. Date: Jul. 27, 2006

(54) Assay and process for labeling and detection of micro rna and small interfering rna sequences

(76) Inventors: Richard Joseph, Stoughton, MA (US); James DiMeo, Needham, MA (US)

Correspondence Address: GIFFORD, KRASS, GROH, SPRINKLE, ANDERSON;& CITKOWSKI, P.C., P.O. BOX 7021, TROY, MI, 48007-7021, US

(21) Appl. No.: 10/563,347

(22) PCT Filed: Jul. 2, 2004
(86) PCT No.: PCT/US04/21439

§ 371 Date: Mar. 28, 2006

(30) Foreign Application Priority Data
Jul. 2, 2003 (US) 60484579

(51) Int. Cl. C12Q 1/68

(52) U.S. Cl. 435/6

(57) ABSTRACT

A process for protecting a short RNA fragment includes labeling a short RNA fragment with a detectable platinum compound forming a labeled small RNA fragment. Resulting labeled short RNA fragment is exposed to a capture oligonucleotide. The capture oligonucleotide includes at least two replicates of a nucleotide sequence complimentary to the short RNA fragment nucleotide sequence. The labeled short RNA fragment and the captured oligonucleotide sequence are brought into contact under hybridization conditions. With hybridization, the marker moiety is detected on the hybridized labeled small RNA fragment-captured oligonucleotide conjugant. A detection array for short RNA fragment includes a substrate having multiple spots, with a first spot including a first capture oligonucleotide including at least two replicates of a nucleotide sequence complimentary to a first short RNA fragment along with an additional nucleotide sequence functioning as a universal controller or spacer. Another spot on the array includes a second capture oligonucleotide having at least two replicates of a sequence complimentary to a second short RNA fragment along with an additional sequence functioning as a universal controller or spacer. Verified small RNA fragments are also obtained after mobilizing a sequence through elution and optional platinum compound removal therefrom.

### RELATED APPLICATION

This application claims priority of U.S. Provisional Patent Application Ser. No. 60/484,579 filed Jul. 2, 2003, which is incorporated herein by reference.

### FIELD OF THE INVENTION

The present invention relates to an assay and a process for labeling short RNA fragments and the design of an assay method for the detection and binding thereof and, in particular, to a microarray capable of binding labeled short RNA fragments that have been synthesized in vivo.

### BACKGROUND OF THE INVENTION

Only recently has the biological field gained an appreciation for the role of RNA interference (abbrev. RNAi) in gene regulation and mRNA degradation. RNAi mechanisms have now been found in a wide variety of cell types and shown to control expression of genes post-transcriptionally including those genes expressed as a result of viral infection, mutagens and cancers. mRNA degradation has been shown to be responsive to the presence of very, short 21-23 base, double-strand, complementary RNA to preclude translation into functional proteins. S. M. Hammond et al., Nat. Rev. Genet. 2, 110-119 (2001); G. Hutvágner et al., Curr. Opin. Genet. Dev. 12, 225-232 (2002); P. A. Sharp et al., Genes Dev. 15, 485-490 (2001); P. M. Waterhouse et al., Nature_{—}411, 834-842 (2001); G. Hutvágner et al., Science 297, 2056-2060 (2002). The process of RNAi is now known to involve Dicer enzyme that cleaves double-stranded RNA into small RNA fragments. These small RNA fragments are classified as either micro-RNA (miRNA) and small interfering RNA (siRNA) based on their ultimate function or mechanism of regulation. RNAi is brought about by the small RNA fragments degrading the mRNA in the case of siRNA or in the case of mRNA simple binding to the mRNA that codes for a protein sequence through the action of the ribosome. RISC enzyme complex has been implicated in assisting the binding of the small RNA fragments to identify complementary sequence and degrade mRNA. RNAi has also been implicated in modifying gene expression across generations without changes in cellular DNA sequences, commonly referred to as epigenetics. J. Couzin, Science 298, 2296-2297 (2002).

RNAi technology is currently being employed to study specific gene expression in whole animals as an alternative for the older knock-out mutation technology. The ability to specifically modulate specific genes via RNAi in a normal, living organism without needing to produce many animal models/strains each with specific mutations (knock-out genes) opens a new door to the understanding of regulation and interaction of the many complex biochemical pathways found in cells.

RNAi has been proposed as having utility in a variety of genetic based therapeutics including treatment of viral infection, cancer, neurodegenerative disorders, inflammatory disease and autoimmune diseases. T. Tuschl et al., Molecular Interventions 2, 158-167 (2002). The development of a viable therapeutic requires the ability to screen a large number of RNA fragments.

Although there are no satisfactory methods for labeling small RNA fragments, a method of chemical labeling of RNA fragments based on the use of a mustard gas derivative to label in vitro synthesized oligonucleotides has been commercialized for use in intracellular small RNA fragment hybridization and detection. Representative of the conventional labeling scheme is the reagent kit Label-IT® (Mirus Technologies). The mustard gas based labeling system has met with limited success owing to the highly toxic nature of the mustard derivatives, instability of mustard gas reagent, and a marginal detection sensitivity. Thus, there exists a need for a superior chemical labeling agent for small, in vivo synthesized RNA fragments that are capable of binding to an array and readily detected.

The current platform of choice, for example microarrays, for detecting and monitoring levels of RNAi within a cell are also currently being developed and designed. One such type of microarray includes the chemical synthesis, in situ of short, complementary DNA oligo sequences directly upon a glass, microarray substrate. Alternatively, specially modified (e.g., 5′-amino or sulfhydryl modified) DNA oligonucleotides have been directly spotted onto a glass microarray support. Thus, there exists a need for a superior methodology of being able to design user-friendly, flexible methods for spotting short, complementary DNA (or RNA) oligonucleotides to the family of RNAis of interest. The design of the spotted oligonucleotides preferably includes: no requirement for special chemical modifications, a complementary sequence(s) which bind to the RNAi of interest, and a sequence element(s) which could be used as an internal control enabling one to measure either qualitatively or quantitatively variations in expression levels of RNAi species within a cell.

### SUMMARY OF THE INVENTION

A process for detecting a short RNA fragment includes labeling a short RNA fragment with a detectable platinum compound forming a labeled small RNA fragment. A resulting labeled short RNA fragment is exposed to a capture oligonucleotide. The capture oligonucleotide includes at least two replicates of a nucleotide sequence complimentary to the short RNA fragment nucleotide sequence. The labeled short RNA fragment and the captured oligonucleotide sequence are brought into contact under hybridization conditions. With hybridization, the marker moiety is detected on the hybridized labeled small RNA fragment-capture oligonucleotide conjugant.

A detection array for short RNA fragments includes a substrate having a first spot thereon. The first spot includes a first capture oligonucleotide having at least two replicates of a nucleotide sequence complimentary to a first short RNA fragment. The first capture oligonucleotide also includes an additional nucleotide sequence functioning as a universal control or a spacer. A second spot on the substrate is displaced from the first spot and includes a second capture oligonucleotide including at least two replicates of a nucleotide sequence complimentary to a second short RNA fragment. The second capture oligonucleotide also includes an additional nucleotide sequence functioning as a universal control or a spacer.

A detectable short RNA fragment is also disclosed and includes a small RNA fragment bound to a detectable platinum compound. Small RNA fragment immobilized on a detector array is detailed above. The method of detecting a small RNA fragment by binding a detectable platinum compound thereto and exposing the same to a detector array as detailed above is also provided. Similarly, it is appreciated that a purified small RNA fragment is obtained by performing a process as detailed above followed by removal of the platinum compound having a marker moiety.

A commercial package is provided that includes a detector array as described above and a detectable platinum compound together with instructions for the use thereof as a detector for small RNA fragments.

### DETAILED DESCRIPTION OF THE PREFERRED EMBODIMENTS

The present invention has utility in the labeling and detection of short RNA fragments from a variety of sources including in vivo and in vitro syntheses. The labeled short RNA fragments are then hybridized onto a microarray. The labeling compounds contain a fluorophore, a hapten or other marker group and brought into contact with a glass microarray having specially designed spotted capture oligonucleotides bound thereto. The spotted oligonucleotides may include a unique sequence, which acts as an internal control element for the hybridization on the array to permit standardization and quantification. Complementary oligonucleotide(s) are prepared to the control sequences, labeled under conditions similar to the small RNA fragments however the label is uniquely identifiable from the label attached to the small RNA fragments (e.g. two spectrally distinct fluorophores), and mixed with the labeled small RNA fragments prior to hybridization. It is appreciated that in some instances, the mixing of the control sequence oligonucleotide(s) with the small RNA fragments may occur before the labeling process and thus both are labeled with the same identifiable label. Upon exposing the labeled small RNA fragments to the microarray under conditions suitable for hybridization, hybridization events are detected by methods conventional to the art that illustratively include direct fluorescence and signal amplification methodologies such as TSA, or other conventional reporter methods.

As used herein, the term “a short RNA fragment” is defined to be a micro-RNA or small interfering RNA ranging in length from 20 to 28 nucleotides where a micro-RNA is named consistent with the guidelines detailed in Ambros et al., RNA 9:277-279 (2003).

According to the present invention, various types of small RNA fragments are labeled and detected. Suitable sources of RNA operative with the present invention illustratively include cellular isolates, in vitro synthesized oligonucleotides and RNA viruses. In those instances where an RNA sample is believed to include a variety of RNA sequence lengths, it is preferred that those RNA sequences having a length of greater than 80 nucleotides be removed prior to labeling. More preferably, sequences having a length of greater than 50 nucleotides are removed. Purification to remove excess length RNA nucleotide sequences is performed by methods common to the art; these methods illustratively include molecular weight cutoff filters, and electrophoretic migration. It is appreciated that short RNA fragments having certain complementary sequences may associate as an at least in part double-stranded or other associative structures and as such purification molecular weight cutoff limits are adjusted accordingly.

The present invention directly chemically labels short RNA fragments using Universal Labeling System (ULS). The ULS chemical label involves attachment of a platinum based compound to the short RNA fragment where the identity and conditions for affecting short RNA fragment labeling are detailed in U.S. Pat. No. 6,133,038 and U.S. Pat. No. 5,580,990. It is appreciated that the specific probe moiety, stabilizing substituents and detectable marker moieties are dictated by the nature of the short RNA fragments in question and the chosen detection methodology. Detectable marker moieties operative herein illustratively include radioisotope labels; enzymes that create a detectable compound after reaction with a substrate; specific binding pair components such as: avidin and streptavidin binding to biotin, biocytin, or aminobiotin, antibody binding to haptens, for example, but not limited to, anti-DIG:DIG, anti-DNP:DNP or anti-Fluorescein:Fluorescein, or lectins binding to sugars; colloidal dye substances, fluorophores such as fluoresceins, rhodamines, sulforhodamines, cyanines and the like; reducing substances such as eosin, erythrosine, and the like; dyed light latex sols, metal sols, particulate sols, chromophores and other detectable markers known in the art.

It is appreciated that a marker moiety is attached directly to a platinum metal center or through a spacer group. It is further appreciated that a spacer group is highly desirable in instances where steric effects interfere with binding of a target short RNA fragment. Stabilizing substituents include those moieties that are generally stable under conditions of storage and labeling. Suitable stabilizing substituents according to the present invention are chosen to provide a desired compound with respect to properties illustratively including solubility, hydrophobic lipophilic balance, steric bulk, and nonreactivity in the face of subsequent reagents. Preferably, stabilizing substituents are linked to form a bidentate or polydentate ligand capable of occupying two or more ligand sites of the labeled platinum atom. Of the bidentate ligands, aliphatic amine compounds are preferred. Bidentate stabilizing ligands are particularly preferred in conjunction with a platinum (II) label with ethylene diamine being a specific embodiment of a preferred bidentate ligand. The stabilization of a platinum (IV) labeling compound according to the present invention includes monodentate, bidentate and polydentate stabilizing ligands, or a combination of monodentate and bidentate ligands. Diethylene triamine is a specific embodiment of a preferred polydentate stabilizing ligand for a platinum (IV) atom of an inventive labeling dye.

A platinum atom of an inventive label includes in addition to the detectable marker and stabilizing substituents a displaceable leaving group that is substituted by a short RNA fragment under reaction conditions resulting in a stable and detectably labeled short RNA fragment. A leaving group associated with a platinum labeling compound according to the present invention includes any group which allows for the formation of a bond between the platinum atom center of the label and the nucleic acid under a given set of reaction conditions based on the relative electronegativity between the leaving group and the target short RNA fragment. Representative leaving groups operative herein illustratively include fluorine, chlorine, bromine, sulfate, nitrate, phosphate, carbonate, phosphonates, carboxylates, oxalates, citrates, alcohols, monoalkyl sulfoxide, and dialkyl sulfoxides.

Labeling of a short RNA fragment according to the present invention includes introducing a platinum labeling compound having a leaving group to a quantity of short RNA fragment targets in a preferably aqueous solution at a temperature and for a time sufficient to induce reaction. Typical reaction conditions include incubating a sample of target short RNA fragments with a quantity of detectable platinum labeling compound at a temperature from 20° to 70° C. for from about 15 minutes to 24 hours. An exemplary labeling of a sample of target short RNA fragments by a detectable platinum label occurs in deionized water at 65° C. in about 1 hour. It is appreciated that the stoichiometry between the detectable platinum compound label and the quantity of target short RNA fragments is variable. In a preferred embodiment, the label is present in stoichiometric excess relative to the quantity of target short RNA fragments present. Following the labeling reaction, unincorporated detectable platinum compound label is preferably removed by conventional purification techniques illustratively including ultrafiltration, chromatography such as size exclusion chromatography, dialysis, and centrifugation.

The labeled short RNA fragments are then combined with a hybridization buffer and exposed to at least one capture oligonucleotide composed of two or more replicates of a specific capture oligonucleotide sequence. A specific capture oligonucleotide sequence represents at least the 21 to 28 nucleotide bases complementary to a labeled short RNA fragment that is potentially present within the sample and is in solution or immobilized. Preferably, a glass microarray is spotted with multiple capture oligonucleotides that vary in capture sequences therebetween. Preferably, such an array has at least 10 different capture oligonucleotides spotted thereon. More preferably, the glass microarray has at least 100 different capture oligonucleotides spotted thereon. A capture oligonucleotide is immobilized on an inventive glass microarray through conventional techniques and linkages.

In a preferred embodiment of the present invention, an inventive capture oligonucleotide also includes a universal nucleotide control sequence or spacer sequence therein. More preferably, the universal nucleotide control sequence or spacer sequence is interspersed between the at least two specific capture sequences making up the complete capture oligonucleotide. Alternatively, a specific capture sequence is interspersed between the at least two universal nucleotide control sequences making up the complete capture oligonucleotide.

Maintaining a sample of labeled small RNA fragments exposed to a capture oligonucleotide at 37° Celsius for from 18 to 20 hours in a conventional hybridization buffer such as 6× sodium citrate (Molecular Cloning, 2^{nd} Ed., Sambrook et al., B.13) allows for hybridization events to occur. Percent sequence identity between a labeled small RNA fragment and a capture oligonucleotide under these conditions exceeds 82% as calculating according to “Current Methods in Sequence Comparison and Analysis,” Macromolecular Sequencing and Synthesis, Selected Methods and Applications, pp 127-149, 1989, Allen R. Liss, Inc.

Detection of hybridization events is dictated by the identity of the detectable platinum label marker moiety. In the case of a glass microarray, positional detection of a marker signal allows for simultaneous screening of hybridization events across all the spotted capture oligonucleotides. Hybridization event detection is recognized to occur through direct spectroscopic measurement such as fluorescence; radiographic detection; or via signal amplification methods such as TSA subsequent reaction of an enzyme such as horseradish peroxidase, alkaline phosphatase, beta galactosidase, glucose oxidase, luciferase or the like reacting with the substrate therefor; specific binding pair formation as detailed above; or magnetic measurement in the case of a marker having a magnetic signal thereto. In instances where cellular extracts are analyzed for short RNA fragments, it is appreciated that multiple micro-RNAs are often responsible for imparting effective RNAi to a cell. The ability to screen a large number of potential small RNA fragments for effectiveness in precluding exogenous genetic material expression requires identification of both small RNA fragment nucleotide sequence identity and quantity.

Patents and publications mentioned in the specification are indicative of the levels of those skilled in the art to which the invention pertains. These patents and publications are incorporated herein by reference to the same extent as if each individual patent or publication was specifically and individually incorporated herein by reference.

The foregoing description is illustrative of particular embodiments of the invention, but is not meant to be a limitation upon the practice thereof.

1. A process for detecting a short RNA fragment comprising:
labeling the short RNA fragment having a nucleotide sequence with a detectable platinum compound having a marker moiety to form a labeled small RNA fragment;
exposing said labeled short RNA fragment to a capture oligonucleotide comprising at least two replicates of a nucleotide sequence complementary to the nucleotide sequence of said short RNA fragment;
contacting said labeled short RNA fragment and said capture oligonucleotide to hybridization conditions; and
detecting the marker moiety upon hybridization between said labeled small RNA fragment and said capture oligonucleotide.

2. The process of claim 1 wherein said small RNA fragment is present in a mixture of in vivo synthesized RNA fragments.

3. The process of claim 1 wherein said marker moiety is selected from the group consisting of: a fluorophore, a hapten, a radioisotope, an enzyme, an enzyme substrate, a dye, a sol, a chromophore, and an antibody.

4. The process of claim 1 wherein said capture oligonucleotide is immobilized on a solid substrate.

5. The process of claim 4 wherein said solid substrate is a microarray spotted with said capture oligonucleotide and a plurality of different capture oligonucleotides that vary in nucleotide sequence relative to said capture oligonucleotide.

6. The process of claim 1 wherein said capture oligonucleotide further comprises an additional nucleotide sequence having a function selected from the group consisting of: universal control, a spacer, and a combination thereof.

7. The process of claim 6 wherein said additional nucleotide sequence is interspersed between said at least two replicates.

8. The process of claim 6 wherein at least two additional nucleotide sequences surround the complementary RNA nucleotide sequence of interest.

9. The process of claim 1 wherein hybridization conditions include heating said labeled short RNA fragment and said capture oligonucleotide to between 30° and 40° Celsius.

10. The process of claim 1 wherein detection of hybridization between said labeled short RNA fragment and said capture oligonucleotide is by fluorescence.

11. The process of claim 1 wherein detection of hybridization between said labeled short RNA fragment and said capture oligonucleotide is by signal amplification.

12. The process of claim 11 wherein the signal amplification is tyramide signal amplification.

13. The process of claim 1 further comprising the step of removing nucleotide sequences over 80 nucleotides in length prior to labeling.

14. The process of claim 1 further comprising the step of purifying said labeled short RNA fragment prior to exposure of said labeled short RNA fragment to said capture oligonucleotide.

15. A detection array for short RNA fragments comprising:
a substrate;
a first spot on said substrate comprising a first capture oligonucleotide having at least two replicates of a nucleotide sequence complementary to a first short RNA fragment and having an additional nucleotide sequence having a function selected from the group consisting of: universal control and spacer; and
a second spot on said substrate displaced from said first spot comprising a second capture oligonucleotide having at least two replicates of a nucleotide sequence complementary to a second short RNA fragment and having an additional nucleotide sequence having a function selected from the group consisting of: universal control and spacer.

16. The array of claim 15 wherein said substrate is glass.

17. The array of claim 15 wherein said plurality of spots includes at least 10 spots.

18. The array of claim 15 wherein said first spot has a linear dimension of from 1 to 100 microns.

19. The array of claim 15 wherein the additional nucleotide sequence of said first capture oligonucleotide is interspersed between the at least two replicates.

20. A detectable small RNA fragment comprising a small RNA fragment bound to a detectable platinum compound, said small RNA fragment immobilized on a detector array according to claim 15 or 16.

21. A method of detecting a small RNA fragment which comprises binding a detectable platinum compound to said small RNA fragment and exposing the same to a detector array of claim 15.

22-23. (canceled)

24. A commercial package comprising a detector array according to claim 15 and a detectable platinum compound together with instructions for the use thereof as a detector for small RNA fragments.

25-26. (canceled)
